(19) United States
(12) Patent Application Publication
(10) Pub. No.: US 2016/0008119 A1
(43) Pub. Date: Jan. 14, 2016

(54) TUBE ASSEMBLY

(71) Applicant: TERUMO KABUSHIKI KAISHA, Tokyo (JP)

(72) Inventors: Masakatsu KAWAURA, Sunnyvale, CA (US); Shigeki ARIURA, Ebina-shi (JP); Nao YOKOI, Sunnyvale, CA (US)

(73) Assignee: TERUMO KABUSHIKI KAISHA, Tokyo (JP)

(21) Appl. No.: 14/865,016

(22) Filed: Sep. 25, 2015

Related U.S. Application Data
(63) Continuation of application No. PCT/JP2013/059889, filed on Apr. 1, 2013.

(51) Int. Cl. A61F 2/00; A61B 17/06; A61B 17/34

(52) U.S. Cl. CPC A61F 2/0045; A61B 17/3403; A61B 17/3468; A61B 17/06109; A61B 2017/00805

(57) ABSTRACT

A tube assembly is disclosed which includes an elongated medical tube and a medical elongated body insertable in the medical tube and higher than the medical tube in rigidity and which is used in an assembled state with the medical elongated body inserted in the medical tube to be assembled together. The tube assembly further includes a puncturing section adapted to puncture a living body; a dissecting section adapted to dissect the living body as the puncturing section punctures the living body; and a rotation restraining section adapted to restrain the medical tube from rotating about its axis in relation to the medical elongated body in the assembled state.

### CROSS-REFERENCES TO RELATED APPLICATIONS

This application is a continuation of International Application No. PCT/JP2013/059889 filed on Apr. 1, 2013, the entire content of which is incorporated herein by reference.

### TECHNICAL FIELD

The present disclosure generally relates to a tube assembly.

### BACKGROUND DISCUSSION

If a person suffers from a urinary incontinence, for example, if a person suffers from a stress urinary incontinence, then urine leakage can occur when abdominal pressure is applied during normal exercise or by laughing, coughing, sneezing or the like. The can be caused, for example, by the fact that the pelvic floor muscle which is a muscle for supporting the urethra is loosened by birth or the like.

For the treatment of urinary incontinence, a surgical treatment is effective, in which there is used, for example, a tape-shaped implant called “sling.” The sling is indwelled inside the body and the urethra is supported by the sling (see, for example, Japanese Patent Laid-open No. 2010-99499). In order to indwell the sling inside the body, an operator would incise the vagina with a surgical knife, dissect the biological tissue between the urethra and vagina, and make the dissected region and the outside communicate with each other through obturator foramens by use of a puncture needle or the like. Then, in this condition, the sling is indwelled into the body.

When dissecting the biological tissue, however, the degree of the dissection may be excessively high or may be insufficient. For example, if the degree of dissection is excessively high, the sling is instable upon being placed indwelling, and is therefore unable to support the urethra satisfactorily. When the degree of dissection is insufficient, for example, the sling is placed indwelling in a shrunken state; in which case, the sling may not be able to satisfactorily support the urethra.

### SUMMARY

A tube assembly is disclosed by which an insertion hole having a required minimum size for indwelling of an implant in a living body can be formed in the living body relatively easily and reliably.

According to an exemplary embodiment, a tube assembly can include an elongated medical tube and a medical elongated body insertable in the medical tube and higher than the medical tube in rigidity, and is used in an assembled state with the medical elongated body inserted in the medical tube to be assembled together. The tube assembly further can include a puncturing section adapted to puncture a living body, a dissecting section adapted to dissect the living body as the puncturing section punctures the living body; and a rotation restraining section adapted to restrain the medical tube from rotating about its axis in relation to the medical elongated body in the assembled state.

According to an exemplary embodiment, the medical tube has a distal opening where a distal end of the medical tube opens, and the medical elongated body has a sharp needle tip at a distal end of the medical elongated body, the needle tip protruding from the distal opening to function as the puncturing section.

According to an exemplary embodiment, the tube assembly preferably has a configuration wherein the medical elongated body has an elongated body side outside diameter gradually increasing section where its outside diameter gradually increases along a proximal direction from the needle tip, and the elongated body side outside diameter gradually increasing section functions as the dissecting section.

According to an exemplary embodiment, the medical elongated body may be flat shaped in cross section at least at the elongated body side outside diameter gradually increasing section.

According to an exemplary embodiment, the tube assembly preferably has a configuration wherein the medical tube has a sharp needle tip at a distal end of the medical tube, and the needle tip functions as the puncturing section.

According to an exemplary embodiment, the medical tube has a tube-side outside diameter gradually increasing section where its outside diameter gradually increases along a proximal direction from a distal end of the medical tube, and the tube-side outside diameter gradually increasing section functions as the dissecting section.

According to an exemplary embodiment, the medical tube may be flat shaped in cross section at least at the tube-side outside diameter gradually increasing section.

According to an exemplary embodiment, the tube assembly may further include a movement restraining section which restrains the medical tube from moving in an axial direction of the medical tube in relation to the medical elongated body in the assembled state.

According to an exemplary embodiment, a proximal portion of the medical tube may be adapted to make contact with an extension section extended from the medical elongated body, thereby exhibiting a function as the movement restraining section.

According to an exemplary embodiment, the restraint by the movement restraining section can be released.

According to an exemplary embodiment, the tube assembly preferably has a configuration wherein each of the medical tube and the medical elongated body is formed, at least at part of the medical tube and the medical elongated body in a longitudinal direction, with a flat section which is flat shaped in cross section, and the flat sections of the medical tube and the medical elongated body overlap with each other in the assembled state so as to exhibit a function as the rotation restraining section.

According to an exemplary embodiment, each of the medical tube and the medical elongated body has a curved section where at least part of the medical tube and the medical elongated body in a longitudinal direction is curved in a circular arc shape, and the curved sections of the medical tube and the medical elongated body overlap with each other in the assembled state so as to exhibit a function as the rotation restraining section.

According to an exemplary embodiment, the tube assembly is preferably so configured that the direction in which the dissecting section dissects the living body is mainly a direction inclined against a center axis of the curved sections.

According to an exemplary embodiment, the tube assembly may further include an implant; and an implant package having a wrapping material, the wrapping material including a bag-shaped wrapping material main body adapted to accommodate the implant therein and a flexible linear body for pulling the wrapping material main body.

According to an exemplary embodiment, an implant has a stopper adapted to make contact with a surface of the living body when the implant is indwelled in the living body.

According to an exemplary embodiment, the tube assembly may be used for treating a disease in a pelvic organ by indwelling an implant between a urethral lumen and a vaginal cavity.

According to the described aspect of the present disclosure, at the time of indwelling an implant into a living body, to form in the living body an insertion hole in and through which the implant can be inserted and passed. The insertion hole has a required minimum size for stable indwelling of the implant, that is, the insertion hole has a necessary and sufficient size for stable indwelling of the implant. When the implant is inserted into and passed through the insertion hole formed in this way, the implant is prevented from shrinking in the width direction thereof, is put in the state of being sufficiently developed, and is thereby left indwelling stably.

A method is disclosed of forming a path in living body tissue with a tube assembly, the tube assembly including an elongated medical tube and a medical elongated body insertable in the medical tube and higher than the medical tube in rigidity and which is used in an assembled state with the medical elongated body inserted in the medical tube to be assembled together, the method comprising: puncturing a living body with a puncture section of the medical tube; dissecting the living body as the puncturing section punctures the living body with a dissecting section of the medical tube; and restraining the medical tube from rotating about its axis in relation to the medical elongated body in the assembled state with a rotation restraining section.

Where the tube assembly of the present disclosure is used for treating female urinary incontinence, for example, the urethra can be satisfactorily supported from the vagina side by the implant inserted and passed in the insertion hole, and, accordingly, the treatment of female urinary incontinence can be performed in a reliable manner.

### BRIEF DESCRIPTION OF THE DRAWINGS

FIG. 1 is a side view showing a medical device including a tube assembly in accordance with a first exemplary embodiment of the present disclosure;

FIG. 2 is a sectional view taken along line II-II of FIG. 1;

FIG. 3 is a view (as viewed from the direction of arrow A in FIG. 1) for explaining an operating procedure of the medical device shown in FIG. 1;

FIG. 4 is a view (as viewed from the direction of arrow A in FIG. 1) for explaining the operating procedure of the medical device shown in FIG. 1;

FIG. 5 is a view (as viewed from the direction of arrow A in FIG. 1) for explaining the operating procedure of the medical device shown in FIG. 1;

FIG. 6 is a view (as viewed from the direction of arrow A in FIG. 1) for explaining the operating procedure of the medical device shown in FIG. 1;

FIG. 7 is a view (as viewed from the direction of arrow A in FIG. 1) for explaining the operating procedure of the medical device shown in FIG. 1;

FIG. 8 is a view (as viewed from the direction of arrow A in FIG. 1) for explaining the operating procedure of the medical device shown in FIG. 1;

FIGS. 9A to 9C are views (showing a process in which a living body is dissected) as viewed from the direction of arrow B in FIG. 4;

FIG. 10 is a sectional view taken along line X-X of FIG. 4;

FIG. 11 is a sectional view taken along line XI-XI of FIG. 8; and

FIG. 12 is a longitudinal sectional view showing a second exemplary embodiment of the tube assembly of the present disclosure.

### DETAILED DESCRIPTION

A tube assembly of the present disclosure will be described in detail below, referring to preferred embodiments thereof illustrated in the attached drawings.

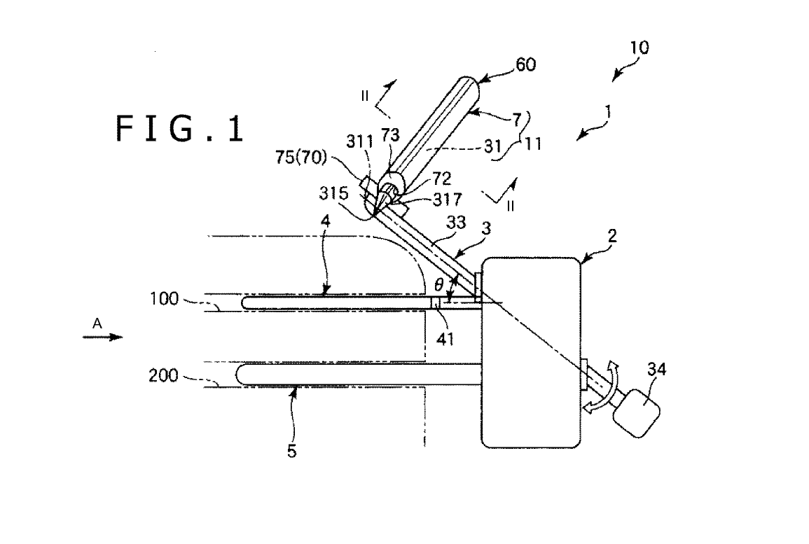

In the following description, the side of a needle tip will be referred to as “distal” side, and the opposite side as “proximal” side. Besides, the upper side in FIGS. 1, 3 to 8, 10 and 11 will be referred to as “upper (upper side)” and the lower side as “lower (lower side).”

A medical device 10 shown in FIGS. 1 and 3 to 8 is for use in treatment of a disorder in a pelvic organ, for example, treatment of female urinary incontinence. In this treatment, an implant (in-body indwelling instrument) 8 is indwelled (embedded) between a urethra (urethral lumen) 100 and a vagina (vaginal cavity) 200. The medical device 10 (tube assembly 11) can include a puncture device 1 and a sheath (medical tube) 7, and, prior to indwelling of the implant 8 in a living body, the medical device 10 forms in the living body an insertion hole 500 into and through which the implant 8 can be inserted and passed. The configuration of each component will be described later.

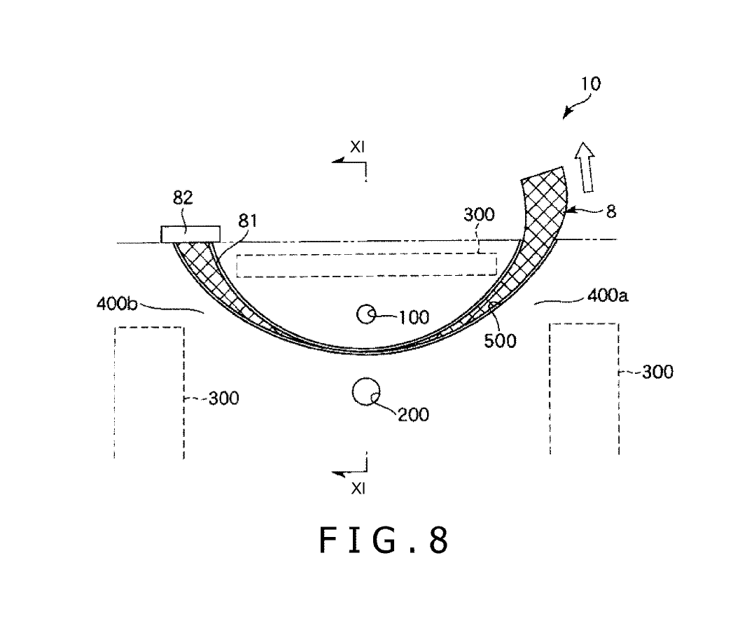

The implant 8 is an embeddable instrument which is called “sling” and which is used for treatment of female urinary incontinence, specifically, for supporting the urethra 100. For example, the implant 8 is an instrument, which, when the urethra 100 is going to move toward the vagina 200 side, supports the urethra 100 so as to restrict its movement in the direction for coming away from the vagina 200. As shown in FIG. 8, the implant 8 can include a belt-shaped flexible implant main body 81, and a stopper 82 fixed to a distal portion (one end portion) of the implant main body 81.

The implant main body 81 is not particularly limited. For example, the implant main body 81 may be composed of a network (lattice)-formed knitted body knitted by causing linear elements to intersect, specifically, network-formed braiding. Examples of the linear element include those, which are circular in cross section, and those which are flat shaped in cross section, for example—belt-shaped (ribbon-shaped) ones. The material constituting the implant main body 81 is not specifically restricted. Examples of the material usable here include various resin materials which are biocompatible. Note that the implant main body 81 is naturally not limited to the network-formed one.

The stopper 82 can include a rigid member, which is greater than the implant main body 81 in width. The stopper 82 is a member which makes contact with a body surface in a condition where the implant 8 is indwelled in a living body, and which prevents the implant 8 from being drawn out of the living body even if the implant 8 is pulled toward the proximal side. The material constituting the stopper 82 is not particularly limited. Examples of the material applicable here include various resin materials. Note that the method for fixing the stopper 82 to the implant main body 81 is not specifically restricted, and, there can be used, for example, a method of fixing by welding (heat welding, high-frequency welding, ultrasonic welding or the like), and a method of fixing by adhesion (adhesion by use of an adhesive or solvent).

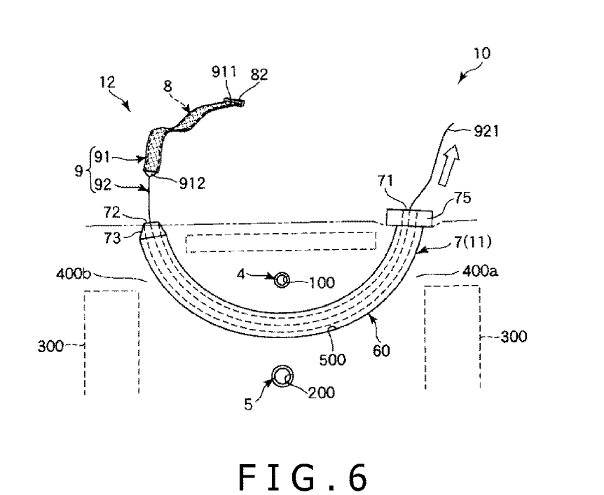

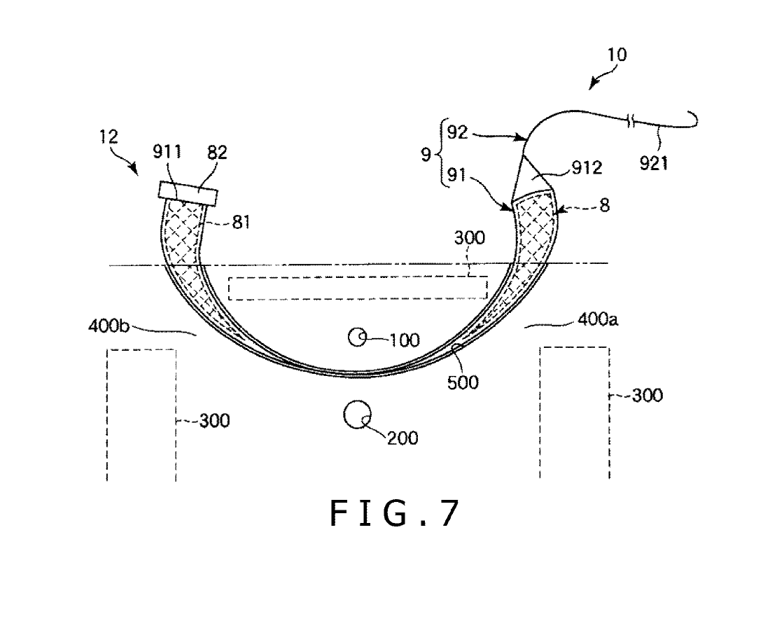

As depicted in FIGS. 6 and 7, the implant 8 has the implant main body 81 accommodated in a wrapping material 9. The wrapping material 9 can include a wrapping material main body 91, which is elongated and flexible, and a string 92 fixed to a proximal portion 912 of the wrapping material main body 91.

The wrapping material main body 91 is a bag-shaped member which is opening at a distal portion 911 thereof and is closed at the proximal portion 912 thereof. The wrapping material main body 91 has a length greater than the length of the implant main body 81, and a width greater than the width of the implant main body 81, which helps ensures that the implant main body 81 is prevented from shrinking inside the wrapping material main body 91 and remains in a sufficiently developed state. In accordance with an exemplary embodiment, the width of the proximal portion 912 of the wrapping material main body 91 gradually decreases along the proximal direction. The material constituting the wrapping material main body 91 is not particularly limited, and, for example, various resin materials can be used as the material.

In the medical device 10, an implant package 12 is preliminarily prepared in which the implant 8 (implant main body 81) is accommodated in the wrapping material 9 (wrapping material main body 91). The implant package 12 is, when inserted into the sheath 7, pulled by the string 92 fixed to the wrapping material main body 91. Note that the string 92 is longer than the overall length of the sheath 7. In addition, a guide wire or a cord-shaped elastic element may also be used, in place of the string 92.

As shown in FIGS. 1 and 3, the puncture device 1 can include a puncture member 3 including a puncture needle (medical elongated body) 31 adapted to puncture a living body (biological tissue), a shaft section 33, and an interlock section 32 interlocking the puncture needle 31 and the shaft section 33; an elongated urethral-insertion member 4 to be inserted into the urethra 100; an elongated vaginal-insertion member 5 to be inserted into the vagina 200; and a support member (restraining means) 2 supporting the puncture member 3, the urethral-insertion member 4 and the vaginal-insertion member 5.

The urethral-insertion member 4, in this embodiment, is supported by the support member 2 in a cantilever fashion. The urethral-insertion member 4 can be a straight hollow or solid body formed from a non-flexible rigid material. Note that in the case where the urethral-insertion member 4 is a hollow body, an end portion on the free end side thereof is preferably opening. In this case, a balloon catheter (not shown) having an expandable and contractible balloon can be inserted into the urethral-insertion member 4. Then, in the condition where the balloon is protruded inside a patient's bladder, the balloon can be expanded. The thus expanded balloon is caught on a bladder neck, whereby the position of the urethral-insertion member 4 relative to the bladder and the urethra 100 can be fixed.

As depicted in FIG. 1, a marker 41 is provided on a peripheral portion of the urethral-insertion member 4. The marker 41 is so disposed that the marker 41 is positioned at a urethral opening at the time when the urethral-insertion member 4 is inserted in the urethra 100 and the end portion of the urethral-insertion member 4 is positioned immediately on the operator's side of the bladder.

The vaginal-insertion member 5, in this embodiment, is supported by the support member 2 in a cantilever fashion. The vaginal-insertion member 5 is straight bar-like in shape. In accordance with an exemplary embodiment, an end portion on the free end side of the vaginal-insertion member 5 is rounded, which helps ensure smooth insertion of the vaginal-insertion member 5 into the vagina 200.

The vaginal-insertion member 5 is disposed on the lower side of the urethral-insertion member 4, at a predetermined distance from the urethral-insertion member 4, in such a manner that its axis is parallel to the axis of the urethral-insertion member 4.

Note that the materials constituting the urethral-insertion member 4, the vaginal-insertion member 5, and the support member 2 are not particularly limited, and, for example, various resin materials and the like can be used for these members.

The puncture member 3 has its shaft section 33 (which serves as a rotating shaft of the puncture member 3) turnably disposed on the support member 2.

The shaft section 33 penetrates the support member 2, and is inhibited from moving in the axial direction relative to the support member 2.

In addition, at an end portion of the shaft section 33, a grip section 34 is provided as an operating section which is operated to turn the puncture member 3. In this embodiment, the grip section 34 is rectangular parallelepiped in shape. At the time of turning the puncture member 3, the grip section 34 is grasped with fingers, and is turned in a predetermined direction. Note that the shape of the grip section 34 is naturally not limited to the just-mentioned.

As shown in FIG. 1, the shaft section 33 is inclined against the axis of the urethral-insertion member 4 so that the distance between the axis of the shaft section 33 and the axis of the urethral-insertion member 4 increases toward the left side in FIG. 1. The inclination angle θ can be, for example, preferably 20 to 60 degrees, more preferably 30 to 45 degrees, and further preferably 35 to 40 degrees, which helps ensure that puncture by the puncture needle 31 can be easily carried out and a shorter puncture distance can be realized.

In accordance with an exemplary embodiment, with the inclination angle θ set within the above-mentioned range, the puncture needle 31 can capture left and right obturator foramens 400a and 400b of the pelvis wider on a planar basis, and a wider puncture space for the puncture needle 31 can be secured. For example, in a condition where the patient is set in a predetermined position (lithotomy position), the puncture needle 31 can be made to puncture the biological tissue in a direction comparatively nearer to a perpendicular direction relative to the left and right obturator foramens 400a and 400b of the pelvis. Therefore, the puncture by the puncture needle 31 can be performed relatively easily. In addition, with the puncture needle 31 made to puncture the biological tissue in a direction comparatively nearer to a perpendicular direction relative to the obturator foramens 400a and 400b, the puncture needle 31 passes a shallow portion of tissue, so that a needle tip 315 of the puncture needle 31 can pass between the left and right obturator foramens 400a and 400b while taking the course of a shorter distance. Since the puncture needle 31 is made to pass those zones in the obturator foramens 400a and 400b which are comparatively nearer to the pubic symphysis, preferably safety zones, it is possible to safely puncture a region where there are few nerves and blood vessels which should be prevented from being damaged. Consequently, the puncturing procedure can be less invasive, and a lighter burden on the patient can be realized. Thus, with the inclination angle θ set within the above-mentioned range, the puncture of the patient by the puncture needle 31 can be performed suitably. If the inclination angle θ is below the above-mentioned lower limit or above the above-mentioned upper limit, for example, there may arise a situation, depending on the individual differences concerning the patient or the posture of the patient during the procedure, in which the puncture needle 31 cannot capture the obturator foramens 400a and 400b wide on a planar basis or in which a sufficiently short puncture route cannot be realized. Therefore, it is preferable to make the puncture needle 31 puncture the biological tissue perpendicularly in relation to the left and right obturator foramens 400a and 400b of the pelvis.

By puncturing at the aforementioned angle, it is made easy to aim at a tissue present between a middle-part urethra (which refers to a middle part in the longitudinal direction of the urethra 100) and the vagina 200. The region between the middle-part urethra and the vagina 200 is a position suitable for treatment of urinary incontinence by embedding the implant 8 therein. More preferably, puncturing is conducted in a condition where the urethra 100 or the vagina 200 or both of the urethra 100 and the vagina 200 have been positionally shifted in the manner of being pushed toward the inner side of the body, whereby it is relatively ensured that puncturing between the middle-part urethra and the vagina 200 can be easily achieved. Means for pushing either one of the urethra 100 and the vagina 200 toward the inner side of the body may be, for example, as follows. After the urethral-insertion member 4 and/or the vaginal-insertion member 5 has been inserted into an appropriate position, and before puncturing, the urethral-insertion member 4 and/or the vaginal-insertion member 5 is moved toward the inner side of the body along its axis to a predetermined position. In this case, if the urethral-insertion member 4 and/or the vaginal-insertion member 5 can be provided with a visible marker or a marker recognizable on a non-invasive intracorporeal-image monitor based on X-rays, or ultrasound, the distance the relevant member is moved can be recognized.

In a condition where at least one of the urethra 100 and the vagina 200 is positionally shifted in the manner of being pushed toward the inner side of the body, the puncture needle 31 is made to puncture the biological tissue perpendicularly in relation to the left and right obturator foramens 400a and 400b of the pelvis, whereby a passage can be formed in a position suitable for indwelling the implant 8 therein.

In accordance with an exemplary embodiment, it can be preferable to adopt a setting such that the trajectory of the puncture needle 31 passes the safety zones in the left and right obturator foramens 400a and 400b of the pelvis, to positionally shift at least one of the urethra 100 and the vagina 200 toward the inner side of the body so that the trajectory is located between the middle-part urethra and the vagina 200, and to make the puncture needle 31 puncture along the trajectory, thereby forming a passage.

The puncture needle 31 is elongated in shape, and has the sharp needle tip 315 at the distal end thereof. By the needle tip 315, a living body can be punctured. Thus, the needle tip 315 functions as the puncturing section for puncturing a living body.

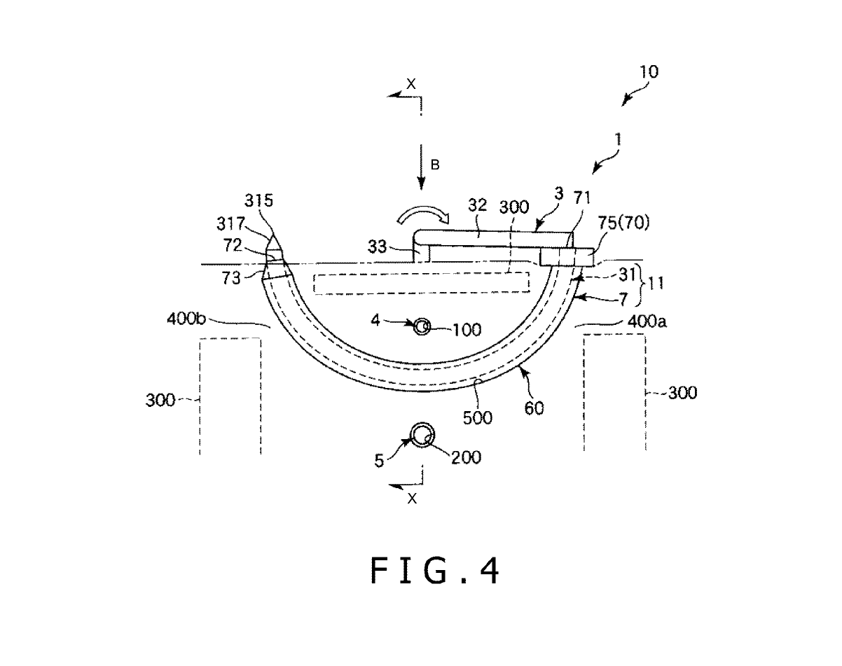

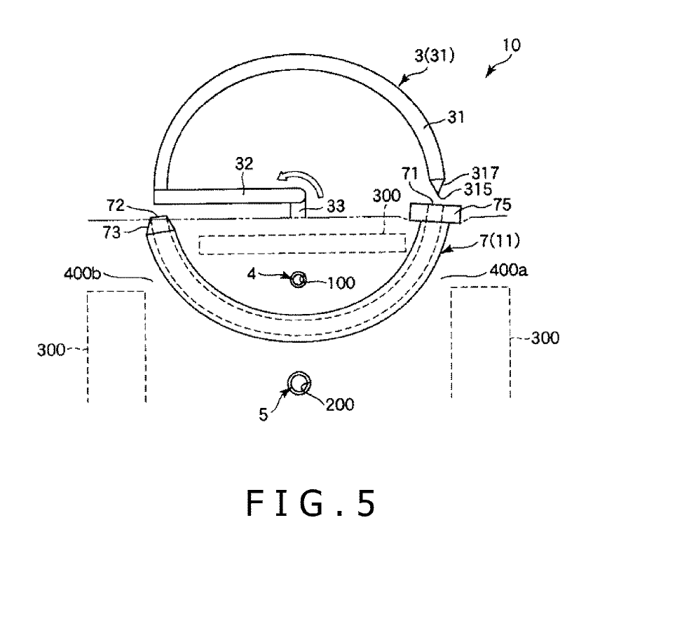

The puncture needle 31 can be inserted into and removed out of the sheath 7 (see FIGS. 3 to 5). At the time of puncturing a living body, the medical device 10 is used as the tube assembly 11 in the assembled state (see FIGS. 3 and 4) with the puncture needle 31 inserted in the sheath 7 to be assembled together. Note that in the assembled state the needle tip 315 is protruding from a distal opening 72 of the sheath 7, and a living body can be reliably punctured by the thus protruding needle tip 315. After puncturing of the living body, the tube assembly 11 is put into a disassembled state (see FIG. 5) with the puncture needle 31 drawn out of the sheath 7.

As shown in FIGS. 1 and 3 to 5, the puncture needle 31 is a body (curved section) which is curved into a circular arc centered on the shaft section 33.

The axis of the puncture needle 31 and the axis of the shaft section 33 are in a positional relation of skew lines, which helps ensures that when the puncture member 3 is turned, the needle tip 315 of the puncture needle 31 is moved in a plane orthogonal to the axis of the shaft section 33, for example, a plane to which the axis of the shaft section 33 is normal, along the circular arc. Note that in this embodiment, the puncture needle 31 is disposed at a position corresponding to an intermediate portion in the longitudinal direction of the urethral-insertion member 4, in the axial direction of the urethral-insertion member 4.

While the needle tip 315 of the puncture needle 31 is oriented clockwise in FIGS. 3 to 5 in this embodiment, this is not restrictive, and the needle tip 315 may be oriented counterclockwise in the figures.

In accordance with an exemplary embodiment, the supporting member 2 can regulate the positional relation of the puncture member 3 and the urethral-insertion member 4 so that when the puncture member 3 is turned to puncture a living body, the needle tip 315 of the puncture needle 31 passes the farther side from a center 311 of the puncture needle 31 than the urethral-insertion member 4 or an extension line thereof, for example, the lower side of the urethral-insertion member 4 or the extension line thereof. Note that the center 311 of the puncture needle 31 is the center of the circular arc of the puncture needle 31, for example, the center of turning of the puncture needle 31 (puncture member 3).

Further, the support member 2 regulates the positional relation of the puncture member 3 and the vaginal-insertion member 5 so that the needle tip 315 of the puncture needle 31 does not collide on the vaginal-insertion member 5 or the extension line thereof when the puncture member 3 is turned to puncture the living body.

In accordance with an exemplary embodiment, for example, the support member 2 can regulate the positional relation of the puncture member 3 and the urethral-insertion member 4 and the vaginal-insertion member 5 so that when the puncture member 3 is turned to puncture the biological tissue, the needle tip 315 of the puncture needle 31 passes between the urethral-insertion member 4 or the extension line thereof and the vaginal-insertion member 5 or the extension line thereof.

This can help ensure that the living body can be punctured by the puncture needle 31 while avoiding the urethra 100 and the vagina 200. Consequently, puncture of the urethra 100 by the puncture needle 31 and puncture of the vagina 200 by the puncture needle 31 can both be prevented.

In addition, since the trajectory of the needle tip 315 of the puncture needle 31 is definite, the operator himself/herself can be prevented from puncturing his/her finger with the puncture needle 31. Thus, safety can be relatively ensured.

The center angle of the circular arc of the puncture needle 31 is not particularly limited, and may be appropriately set according to various conditions. However, the center angle is so set that when puncturing the living body by the puncture needle 31, the puncture needle 31 can enter the body via the body surface on one side of the patient, pass on the lower side of the urethra 100, and go out of the body via the body surface on the other side. In accordance with an exemplary embodiment, for example, the center angle concerning the puncture needle 31 is preferably 150 to 270 degrees, more preferably 170 to 250 degrees, and further preferably 190 to 230 degrees, which can help ensure that when puncturing the living body by the puncture needle 31, the puncture needle 31 can, together with the sheath 7, reliably enter the body via the body surface on one side of the patient, pass on the lower side of the urethra 100, and go out of the body via the body surface on the other side (see FIG. 4).

In accordance with an exemplary embodiment, for example, in this instance, the sheath 7 has its enlarged diameter section 75 making contact with the body surface, whereby further insertion of the sheath 7 into the living body is restrained. Thus, the enlarged diameter section 75 functions as a restricting section for restricting the insertion limit of the sheath 7.

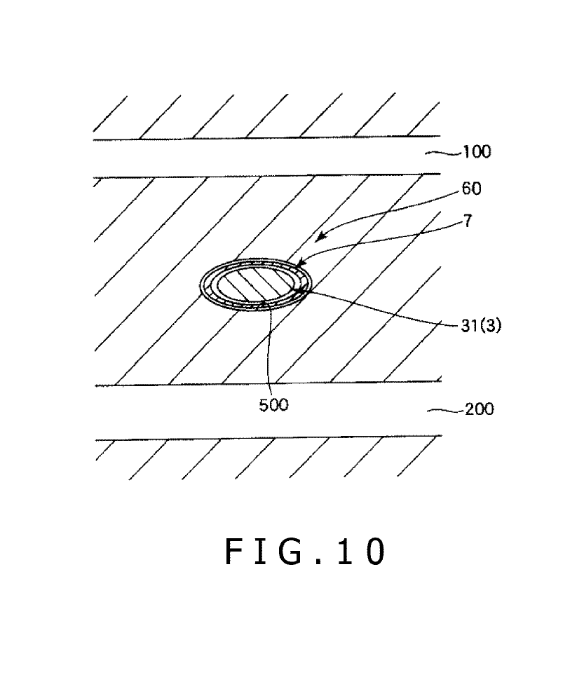

As shown in FIG. 10, the puncture needle 31 is a body (flat section) which is flat shaped in cross section, over the full length thereof.

Note that this flat shape is not particularly limited. For example, the flat shape is preferably an ellipse, as depicted in FIG. 10. Other than the ellipses, there can also be used rhombuses with corners rounded, rectangles (flat shapes) with corners rounded, and spindle-like shapes with a central portion enlarged in width (in diameter) as compared with both end portions.

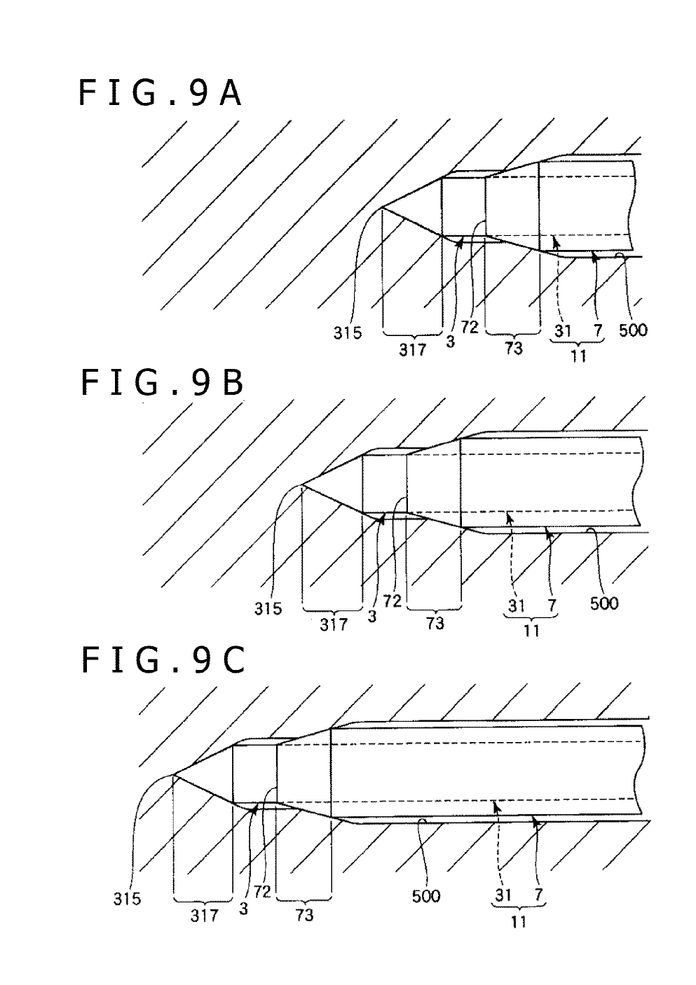

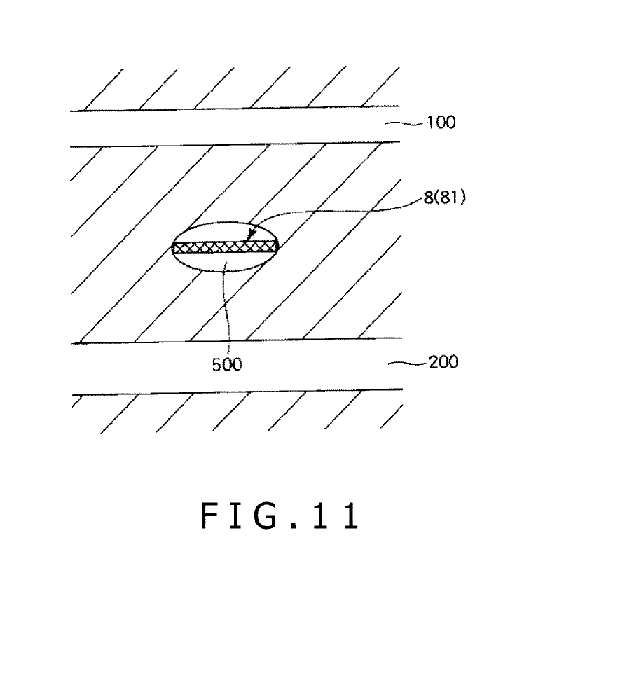

As shown in FIGS. 9A to 9C, the puncture needle 31 is provided at a distal portion thereof with a tapered section (elongated body side outside diameter gradually increasing section) 317 at which the outside diameter gradually increases along the proximal direction from the needle tip 315 (for example, which is tapered). The tapered section 317 functions as a dissecting section which, as the needle tip 315 punctures a living body, dissects the punctured part of the living body so as to gradually expand the punctured part. Since the puncture needle 31 is flat shaped in cross section as aforementioned, naturally, the tapered section 317 is also flat shaped in cross section, and the maximum width thereof is roughly comparable to the width of the implant main body 81 of the implant 8, which helps ensure that at the time of forming the insertion hole 500, the width of the insertion hole 500 can be made to be roughly comparable to the width of the implant main body 81 of the implant 8. Consequently, the implant main body 81 inserted in and passed through the insertion hole 500 is prevented from shrinking within the insertion hole 500, and is put in a sufficiently developed state (see FIG. 11).

As shown in FIGS. 9A to 9C, the direction in which the tapered section 317 dissects the living body is principally the vertical direction in the figures, for example, a direction inclined by the inclination angle θ against the center axis of the curved shape (of being curved into a circular arc shape) of the puncture needle 31, which can help ensure that the urethra 100 can be satisfactorily supported from the vagina 200 side (see FIG. 11).

The puncture needle 31 configured as above may be a solid needle or may be a hollow needle.

In accordance with an exemplary embodiment, the puncture member 3 is higher in rigidity than the sheath 7. The material constituting the puncture member 3 is not specifically restricted. For example, where the sheath 7 is formed from a resin material, examples of the material which can be used for the puncture member 3 include various metallic materials such as stainless steel, aluminum or aluminum alloys, and titanium or titanium alloys.

As shown in FIG. 3, the sheath 7 can include an elongated tubular body, and has the distal opening 72 where a distal end thereof opens and a proximal opening 71 where a proximal end thereof opens. At a peripheral portion of the proximal opening 71, there is formed the enlarged diameter section 75 having an enlarged outside diameter.

Like the puncture needle 31, the sheath 7 is a body (curved section) having a curved shape (of being curved into a circular arc shape) such that the sheath 7 lies along the curved shape (of being curved into a circular arc shape) of the puncture needle 31 in the assembled state.

In accordance with an exemplary embodiment, the overall length of the sheath 7 is smaller than the overall length of the puncture needle 31, which helps ensure that a portion of the puncture needle 31 ranging from the needle tip 315 to a proximal end of the tapered section 317 protrudes from the distal opening 72 in the assembled state, so that puncture by the needle tip 315 and dissection by the tapered section 317 can be performed in a reliable manner.

As shown in FIG. 10, the sheath 7 is also a body (flat section) which is flat shaped in cross section, like the shape of the puncture needle 31 in cross section.

As shown in FIGS. 9A to 9C, the sheath 7 is provided at a distal portion thereof with a tapered section (tube-side outside diameter gradually increasing section) 73 at which its outside diameter gradually increases along the proximal direction from the distal opening 72, for example, which is tapered. The tapered section 73 functions as a dissecting section which, as the needle tip 315 of the puncture needle 31 punctures a living body, dissects the living body in the manner of gradually expanding the living body, following the tapered section 317 of the puncture needle 31.

Note that while the taper angle of the tapered section 73 and the taper angle of the tapered section 317 may be the same, it can be preferable that the taper angles are different as shown in FIGS. 9A to 9C. In this case, for example, the taper angle of the tapered section 73 is preferably smaller than the taper angle of the tapered section 317, which can help ensure that the tube assembly 11 in the assembled state can be smoothly advanced in a living body.

Since the sheath 7 is flat shaped in cross section as aforementioned, naturally, the tapered section 73 is also flat shaped in cross section, and the maximum width of the tapered section 73 is roughly comparable to the width of the implant main body 81 of the implant 8 (is slightly greater than the maximum width of the tapered section 317). The tapered section 73 configured in this way helps ensure that an expanded state of the insertion hole 500 dissected and expanded by the tapered section 317 of the puncture needle 31 is securely maintained, that is, the insertion hole 500 is securely expanded to the same degree as the width of the implant main body 81 of the implant 8.

As shown in FIGS. 9A to 9C, like in the case of the tapered section 317, the direction in which the tapered section 73 dissects a living body is principally the vertical direction in the figures, for example, a direction inclined by the inclination angle θ against the center axis of the curved shape (of being curved into a circular arc shape) of the puncture needle 31.

When the implant main body 81 of the implant 8 is inserted and passed in the insertion hole 500 expanded securely, the implant main body 81 is prevented from shrinking in the width direction thereof, is put in a sufficiently developed state and is set indwelling stably (see FIG. 1). As a result, the urethra 100 can be satisfactorily supported from the vagina 200 side, and, accordingly, treatment of female urinary incontinence can be reliably carried out.

The material constituting the sheath 7 is not particularly limited; for example, various resin materials can be used.

As shown in FIG. 3, in the assembled state, the enlarged diameter section 75 of the sheath 7 is in contact with the interlock section 32 (extension section) extended from the puncture needle 31. This reliably restrains the sheath 7 from unintentionally moving in the axial direction thereof in relation to the puncture needle 31, that is, reliably restrains shifting or slippage of the sheath 7 in the proximal direction. Then, with the puncture member 3 rotated, the puncture needle 31 can be assuredly pushed into a living body together with the sheath 7.

Thus, in the medical device 10, with the enlarged diameter section 75 of the sheath 7 put in contact with the interlock section 32 of the puncture member 3, a function as a movement restraining section 70 for restraining unintentional movement of the sheath 7 is exhibited. In accordance with an exemplary embodiment, the restraint by the movement restraining section 70 can be released by pulling the puncture needle 31 proximally, for example, by rotating the puncture member 3 in the reverse direction to the above. This release of the restraint can help enable the puncture needle 31 to be drawn out of the sheath 7.

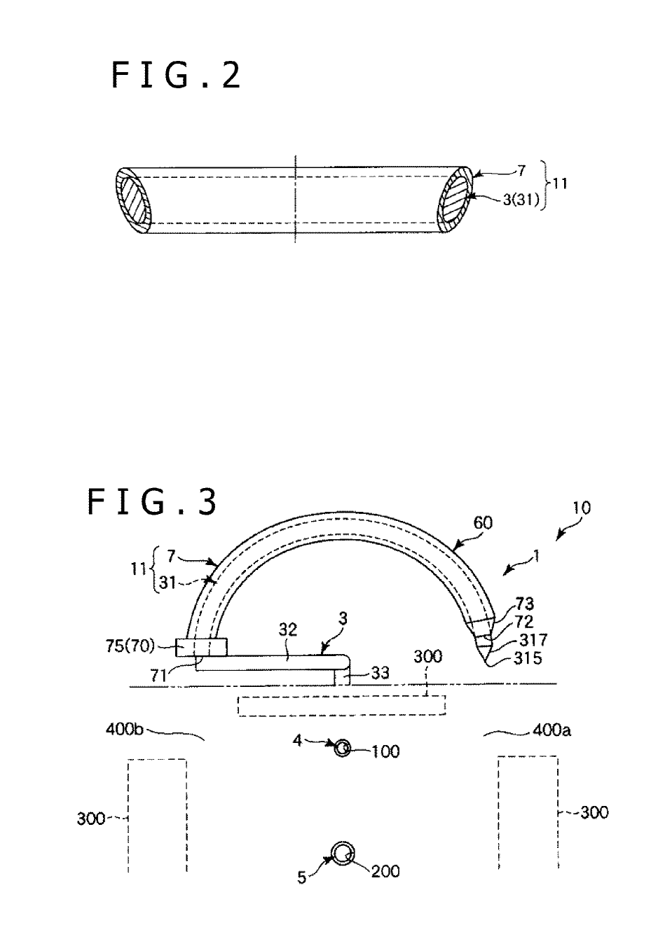

As shown in FIGS. 2 and 10, in the assembled state, the puncture needle 31 flat shaped in cross section and the sheath 7 flat shaped in cross section overlap with each other. This restrains the sheath 7 from rotating about the axis thereof in relation to the puncture needle 31. For example, in the assembled state, the puncture needle 31 curved into a circular arc shape and the sheath 7 curved into a circular arc shape overlap with each other. This overlapping also can restrain the sheath 7 from rotating about its axis relative to the puncture needle 31. This restraint on rotation helps ensure that the direction of dissection (the direction of expansion) by the tapered section 317 of the puncture needle 31 and the direction of dissection (the direction of expansion) by the tapered section 73 of the sheath 7 can be reliably maintained to be the same. As a result, the insertion hole 500 formed by the tube assembly 11 in the assembled state is so configured that the implant main body 81 can be sufficiently developed therein.

Thus, in the medical device 10, owing to the overlapping of the parts flat shaped in cross section and the overlapping of the parts having the curved shapes (of being curved into a circular arc shape), a function as a rotation restraining section 60 for restraining rotation of the sheath 7 is exhibited.

A method of using the medical device 10, for example, a procedure of embedding the implant 8 in a living body, will be described below referring to FIGS. 1 and 3 to 8.

First, as shown in FIGS. 1 and 3, the medical device 10 in the assembled state with the puncture needle 31 inserted in the sheath 7 to be assembled together is mounted onto a patient. In accordance with an exemplary embodiment, the urethral-insertion member 4 of the puncture device 1 is inserted into the patient's urethra 100, and the vaginal-insertion member 5 is inserted into the patient's vagina 200. In this instance, it can be ensured that the marker 41 is positioned at the urethral opening or just on the operator's side of the urethral opening. By this, an end portion of the urethral-insertion member 4 can be positioned just on the operator's side of the bladder.

Next, as shown in FIG. 4, the grip section 34 of the puncture member 3 is gripped, and the puncture member 3 is rotated clockwise in the figure. By this, the needle tip 315 of the puncture needle 31 is moved clockwise in FIG. 4 along the circular arc thereof, to puncture the patient's body surface at an inguinal region on the right side in the figure or near the inguinal region, enter the body, pass an obturator foramen 400a of a pelvis 300, pass on the lower side of the urethra 100, specifically, pass between the urethra 100 and the vagina 200, pass an obturator foramen 400b of the pelvis 300, and protrudes to the exterior of the body via the body surface at an inguinal region on the left side in the figure or near this inguinal region. Attendant on this, the tapered section 317 of the puncture needle 31 and the tapered section 73 of the sheath 7 dissect the living body sequentially and gradually, as aforementioned. As a result, the patient is formed therein with the insertion hole 500 which extends from the body surface at the inguinal region on the right side in FIG. 4 or near the inguinal region, extends through the obturator foramen 400a, then between the urethra 100 and the vagina 200, and through the obturator foramen 400b, to reach the body surface at the inguinal region on the left side in the figure or near this inguinal region. This insertion hole 500 is a penetrating hole expanded to the same degree as the width of the implant main body 81 of the implant 8.

Subsequently, as shown in FIG. 5, the grip section 34 of the puncture member 3 is gripped, and the puncture member 3 is rotated counterclockwise in the figure, with the sheath 7 left in the insertion hole 500, which causes the needle tip 315 of the puncture needle 31 to move counterclockwise in FIG. 5 along the circular arc thereof, enter the patient's body via the body surface at the inguinal region on the left side in the figure or near the inguinal region, pass the obturator foramen 400b of the pelvis 300, pass on the lower side of the urethra 100, specifically, pass between the urethra 100 and the vagina 200, pass the obturator foramen 400a of the pelvis 300, and come out of the body via the body surface at the inguinal region on the right side in the figure or near this inguinal region. In accordance with an exemplary embodiment, for example, the puncture needle 31 is drawn out of the body.

Next, as shown in FIG. 6, the string 92 of the implant package 12 is inserted into the distal opening 72 of the sheath 7 and pushed forward. By this, a proximal portion 921 of the string 92 protrudes from the proximal opening 71 of the sheath 7.

Then, the thus protruding proximal portion 921 of the string 92 is grasped, and the string 92 is pulled. By this, the implant 8 is inserted into and passed through the sheath 7 together with the wrapping material 9.

Subsequently, a distal portion or a proximal portion of the sheath 7 is grasped, and, as shown in FIG. 7, the sheath 7 is pulled, with the implant package 12 left in the insertion hole 500. By this, the sheath 7 is drawn out of the insertion hole 500.

In addition, the puncture device 1 is dismounted from the patient. Specifically, the urethral-insertion member 4 is drawn out of the urethra 100, and the vaginal-insertion member 5 is drawn out of the vagina 200 of the patient.

Next, as shown in FIG. 8, the implant package 12 as a whole is pulled until the stopper 82 makes contact with the living body surface, followed by pulling the wrapping material 9, with the implant 8 left in the insertion hole 500. By this, the wrapping material 9 is drawn out of the insertion hole 500, and the implant 8 is left indwelling in the insertion hole 500. In this instance, the implant main body 81 of the implant 8 is in the state of being sufficiently developed in the width direction thereof. Besides, both sides of the implant main body 81 face toward the urethra 100 side and the vagina 200 side, respectively (see FIG. 11).

Then, the implant 8 is pulled with predetermined forces to adjust the position of the implant 8 relative to the urethra 100, unnecessary portions of the implant main body 81 are cut away, and predetermined treatments are performed, to finish the procedure. As a result, the implant 8 is stably left indwelling in the insertion hole 500, the urethra 100 can be satisfactorily supported from the vagina 200 side, and, accordingly, treatment of female urinary incontinence can be relatively assuredly is accomplished.

Thus, according to the medical device 10, prior to the placement of the implant 8 indwelling in a living body, the insertion hole 500 flat shaped like the implant 8 can be formed relatively easily and reliably in the living body. In addition, the insertion hole 500 is in a required minimum size for stable indwelling of the implant 8 in the insertion hole 500.

In accordance with an exemplary embodiment, according to the medical device 10, surgical incision and dissection at the time of indwelling the implant can be omitted, which have been practiced in the conventional treatment of urinary incontinence. As a result, the burden on the patient can be reduced, that is, low invasive treatment of urinary incontinence can be realized. In addition, the safety of the patient and the operator are relatively high.

In accordance with an exemplary embodiment, the living body can be punctured by the puncture needle 31 while avoiding the urethra 100 and the vagina 200, so that puncturing the urethra 100 or the vagina 200 with the puncture needle 31 can be prevented from occurring, and safety is therefore ensured. In addition, the operator himself/herself can be prevented from puncturing his/her fingertip with the puncture needle 31.

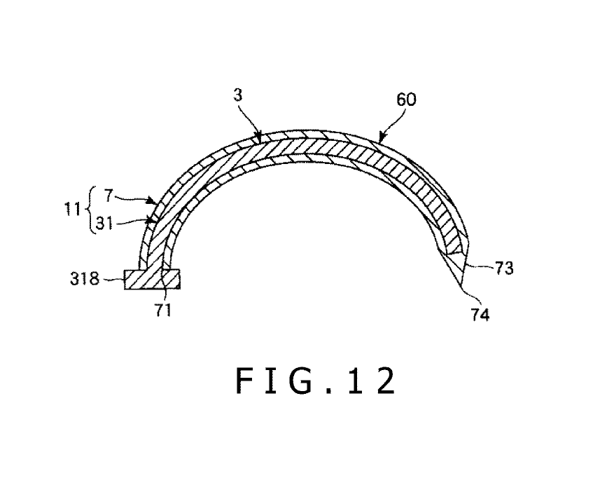

FIG. 12 is a longitudinal sectional view showing a second exemplary embodiment of the tube assembly of the present disclosure.

The second exemplary embodiment of the tube assembly of the present disclosure will be described below referring to this figure. The following description will center on differences from the aforementioned embodiment, and descriptions of the same items as above will be omitted.

This embodiment is the same as the aforementioned first embodiment, except for differences mainly in the configuration (shape) of the sheath.

As shown in FIG. 12, in this embodiment, the distal end of a sheath 7 is closed, and forms a sharp needle tip 74. By this needle tip 74, a living body can be punctured in a reliable manner. Thus, the needle tip 74 can function as a puncturing section for puncturing a living body.

Note that in this embodiment a puncture needle 31 of a puncture member 3 can omit a needle tip 315. The puncture needle 31 configured in this fashion functions as a stylet which reinforces the sheath 7 from inside.

While the tube assembly of the present disclosure has been described with reference to the embodiments illustrated in the drawings, the present disclosure is not limited to the embodiments. Each component of the tube assembly can be replaced with arbitrarily configured one that can exhibit the same or equivalent function to the original. Besides, an arbitrary structure or structures may be added to each of the aforementioned configurations.

The tube assembly of the present disclosure may be a combination of arbitrary two or more configurations (features) of the aforementioned embodiments.

The medical device may have a configuration in which, for example, the vaginal-insertion member is omitted and the restraining means restrains only the positional relation of the puncture needle (puncture member) and the urethral-insertion member.

While the puncture needle of the puncture member is curved in a circular arc shape in its entirety, this is not restrictive. For example, the puncture needle may have a circular arc-shaped curved section, only at part thereof. In accordance with an exemplary embodiment, for example, it can be sufficient for the puncture needle to have a circular arc-shaped curved section at least at part in the longitudinal direction thereof.

It is sufficient for the puncture needle of the puncture member to have a curved section at least at part thereof. For instance, the puncture needle may be curved in an elliptic arc shape in its entirety, or may have an elliptic arc-shaped curved section only at part thereof. Thus, the puncture needle may have an elliptic arc-shaped curved section at least at part thereof.

While the puncture needle of the puncture member is flat shaped in cross section over the full length thereof, this is not restrictive. For example, the puncture needle may be flat shaped in cross section at least at part in the longitudinal direction thereof (tapered section).

While the sheath is curved in a circular arc shape in its entirety, this is not restrictive. For instance, the sheath may have a circular arc-shaped curved section only at part thereof. Thus, it is sufficient for the sheath to have a circular arc-shaped curved section at least at part in the longitudinal direction thereof.

It is sufficient for the sheath to have a curved section at least at part thereof. For example, the sheath may be curved in an elliptic arc shape in its entirety, or may have an elliptic arc-shaped curved section only at part thereof. Thus, the sheath may have an elliptic arc-shaped curved section at least at part thereof.

While the sheath is flat shaped in cross section over the full length thereof, this is not restrictive. For instance, the sheath may be flat shaped in cross section at least at part in the longitudinal direction thereof (tapered section).

While description has been made of a case where the medical device is applied to a device for use in embedding in a living body an embeddable implant for treatment of female urinary incontinence, the use of the medical device is not limited to the described one.

For example, the target of the application of the present disclosure can include excretory disorders attendant on the weakening of the pelvic floor muscle group (urinary urgency, frequent urination, urinary incontinence, fecal incontinence, urinary retention, dysuria or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal stump prolapse, vaginal vault prolapse and the like in which the naming method thereof is based on the prolapsed vaginal-wall part.

Also, overactive tissues include bladder, vagina, uterus, bowel and the like. Lessactive tissues include bones, muscles, fascias, ligaments and the like. In particular, in the case of pelvic floor disorders, the lessactive tissues include an obturator fascia, a coccygeus fascia, a cardinal ligament, an uterosacral ligament, a sacrospinous ligament and the like.

For the procedure for interlocking an overactive tissue with the lessactive tissue in the pelvic floor disorder, there are included a retropubic sling surgery, a transobturator sling surgery (transobturator tape: TOT), a tension-free vaginal mesh (TVM) surgery, a uterosacral ligament suspension (USLS) surgery, a sacrospinous ligament fixation (SSLF) surgery, an iliococcygeus fascia fixation surgery, a coccygeus fascia fixation surgery, and the like.

The tube assembly of the present disclosure can include an elongated medical tube and a medical elongated body insertable in the medical tube and higher than the medical tube in rigidity, and is used in an assembled state with the medical elongated body inserted in the medical tube to be assembled together. The tube assembly further can include: a puncturing section adapted to puncture a living body; a dissecting section adapted to dissect the living body as the puncturing section punctures the living body; and a rotation restraining section adapted to restrain the medical tube from rotating about its axis in relation to the medical elongated body in the assembled state. Therefore, an insertion hole having a required minimum size for stably indwelling of an implant in a living body can be formed in the living body easily and reliably.

The detailed description above describes a tube assembly. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A tube assembly, which includes an elongated medical tube and a medical elongated body insertable in the medical tube and higher than the medical tube in rigidity and which is used in an assembled state with the medical elongated body inserted in the medical tube to be assembled together, comprising:
a puncturing section adapted to puncture a living body;
a dissecting section adapted to dissect the living body as the puncturing section punctures the living body; and
a rotation restraining section adapted to restrain the medical tube from rotating about its axis in relation to the medical elongated body in the assembled state.

2. The tube assembly according to claim 1,
wherein the medical tube has a distal opening where a distal end of the medical tube opens; and
the medical elongated body has a sharp needle tip at a distal end of the medical elongated body, the needle tip protruding from the distal opening to function as the puncturing section.

3. The tube assembly according to claim 2,
wherein the medical elongated body has an elongated body side outside diameter gradually increasing section, wherein the outside diameter gradually increases along a proximal direction from the needle tip, and the elongated body side outside diameter gradually increasing section functions as the dissecting section.

4. The tube assembly according to claim 3,
wherein the medical elongated body is flat shaped in cross section at least at the elongated body side outside diameter gradually increasing section.

5. The tube assembly according to claim 1,
wherein the medical tube has a sharp needle tip at a distal end of the medical tube, and the needle tip functions as the puncturing section.

6. The tube assembly according to claim 1,
wherein the medical tube has a tube-side outside diameter gradually increasing section, wherein the outside diameter gradually increases along a proximal direction from a distal end of the medical tube, and the tube-side outside diameter gradually increasing section functions as the dissecting section.

7. The tube assembly according to claim 6,
wherein the medical tube is flat shaped in cross section at least at the tube-side outside diameter gradually increasing section.

8. The tube assembly according to claim 1, comprising:
a movement restraining section which restrains the medical tube from moving in an axial direction of the medical tube in relation to the medical elongated body in the assembled state.

9. The tube assembly according to claim 8,
wherein a proximal portion of the medical tube is adapted to make contact with an extension section extended from the medical elongated body, and wherein the proximal portion functions as the movement restraining section.

10. The tube assembly according to claim 8,
wherein the restraint by the movement restraining section can be released.

11. The tube assembly according to claim 1,
wherein each of the medical tube and the medical elongated body is formed, at least at part of the medical tube and the medical elongated body in a longitudinal direction, with a flat section which is flat shaped in cross section, and the flat sections of the medical tube and the medical elongated body overlap with each other in the assembled state and function as the rotation restraining section.

12. The tube assembly according to claim 1,
wherein each of the medical tube and the medical elongated body has a curved section where at least part of the medical tube and the medical elongated body in a longitudinal direction is curved in a circular arc shape, and the curved sections of the medical tube and the medical elongated body overlap with each other in the assembled state and function as the rotation restraining section.

13. The tube assembly according to claim 12,
wherein the direction in which the dissecting section dissects the living body is mainly a direction inclined against a center axis of the curved sections.

14. The tube assembly according to claim 1, comprising:
an implant; and
an implant package having a wrapping material, the wrapping material including a bag-shaped wrapping material main body adapted to accommodate the implant therein and a flexible linear body for pulling the wrapping material main body.

15. The tube assembly according to claim 1, comprising:
an implant having a stopper adapted to make contact with a surface of the living body when the implant is indwelled in the living body.

16. The tube assembly according to claim 1,
wherein the tube assembly is used for treating a disease in a pelvic organ by indwelling an implant between a urethral lumen and a vaginal cavity.

17. A method of forming a path in living body tissue with a tube assembly, the tube assembly including an elongated medical tube and a medical elongated body insertable in the medical tube and higher than the medical tube in rigidity and which is used in an assembled state with the medical elongated body inserted in the medical tube to be assembled together, the method comprising:
puncturing a living body with a puncture section of the medical tube;
dissecting the living body as the puncturing section punctures the living body with a dissecting section of the medical tube; and
restraining the medical tube from rotating about its axis in relation to the medical elongated body in the assembled state with a rotation restraining section.

18. The method according to claim 17, comprising:
indwelling an implant between a urethral lumen and a vaginal cavity to treat a disease in a pelvic organ.

19. The method according to claim 17, wherein
the medical tube has a distal opening where a distal end of the medical tube opens;
the medical elongated body has a sharp needle tip at a distal end of the medical elongated body, the needle tip protruding from the distal opening to function as the puncturing section; and
the medical elongated body has an elongated body side outside diameter gradually increasing section, wherein the outside diameter gradually increases along a proximal direction from the needle tip, and the elongated body side outside diameter gradually increasing section functions as the dissecting section.

20. The method according to claim 17, comprising:
forming each of the medical tube and the medical elongated body, at least at part of the medical tube and the medical elongated body in a longitudinal direction, with a flat section which is flat shaped in cross section, and the flat sections of the medical tube and the medical elongated body overlap with each other in the assembled state and function as the rotation restraining section.
